(19) United States
(12) Patent Application Publication
(10) Pub. No.: US 2008/0103312 A1
(43) Pub. Date: May 1, 2008

(54) Processes for the synthesis of 5-phenyl-1-trityl-1H-tetrazole

(76) Inventors: Vinod Kumar Kansal, Haryana (IN); Dhirenkumar N. Mistry, Gujarat (IN); Sanjay L. Vasoya, Gujarat (IN); Vijaykumar D. Lunagariya, Gujarat (IN)

Correspondence Address: KENYON & KENYON LLP, ONE BROADWAY, NEW YORK, NY, 10004, US

(21) Appl. No.: 11/897,173

(22) Filed: Aug. 28, 2007

Related U.S. Application Data
(60) Provisional application No. 60/841,163, filed on Aug. 29, 2006.
(60) Provisional application No. 60/902,079, filed on Feb. 20, 2007.

(51) Int. Cl. C07D 257/04

(52) U.S. Cl. 548/253; 548/250

(57) ABSTRACT

Provided are processes for the synthesis of 5-phenyl-1-trityl-1H-tetrazole, an intermediate useful in the synthesis of irbesartan.

### CROSS-REFERENCE TO RELATED APPLICATIONS

This application claims the benefit of U.S. provisional application Ser. Nos. 60/841,163, filed Aug. 29, 2006; and 60/902,079, filed Feb. 20, 2007, hereby incorporated by reference.

### FIELD OF THE INVENTION

The invention encompasses processes for the synthesis of 5-phenyl-1-trityl-1H-tetrazole, an intermediate useful in the synthesis of irbesartan.

### BACKGROUND OF THE INVENTION

Irbesartan, 2-n-butyl-3-[(2′-(1H-tetrazol-5-yl)-biphenyl-4-yl)methyl]-1,3-diaza-spiro[4,4]non-1-en-4-one, a compound having the chemical structure, 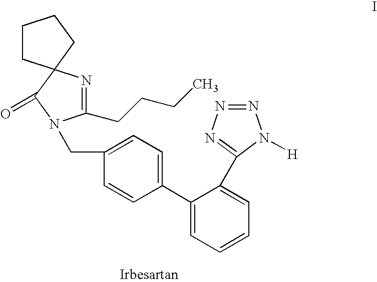
is an antagonist of angiotensin-II receptors or so-called receptors AT-1 and AT-2. Irbesartan is useful in the treatment of cardiovascular diseases such as hypertension, cardiac insufficiency, and cardiac arrhythmia, in the treatment of glaucoma and diabetic retinopathy, and in the treatment of renal insufficiency and diabetic nephropathy.

Irbesartan is marketed under the trade name AVAPRO® by Sanofi Aventis in tablets containing 75 mg, 150 mg, and 300 mg doses of irbesartan.

The preparation of 5-phenyl-1-trityl-1H-tetrazole (“compound II”), 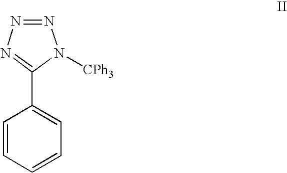
which is a key intermediate in the synthesis of irbesartan, is disclosed in International PCT Publication Nos. WO 94/03435 (“WO '435”), WO 94/11012 (“WO '012”), and WO 2004/065383 (“WO '383”), as well as in U.S. Pat. No. 5,965,738 (“'738 patent”). WO '012, WO '383, and the '738 patent disclose processes for preparing the 5-phenyl-1-trityl-1H-tetrazole by combining 5-phenyl-1H-tetrazole with chlorotriphenylmethane in the presence of a base, such as triethylamine, in an organic solvent, such as methylene chloride, tetrahydrofuran, or acetonitrile. See WO '012, p. 39 (scheme 8); WO '383, p. 11, 1, 10 to p. 12, 1. 2 (example 1b); '738 patent, col. 12, 1. 62 to col. 13, 1. 10 (referential example 5). The 5-phenyl-1-trityl-1H-tetrazole is isolated, for example, by removing the by-product triethylammonium chloride salt from the reaction mixture, evaporating the organic solvent to give a residue, and, optionally crystallizing the 5-phenyl-1-trityl-1H-tetrazole from the residue. See id. The by-product may be removed from the reaction mixture either by filtration or by washing with water and a 10% citric acid solution. See id. WO '383 reports that 5-phenyl-1-trityl-1H-tetrazole is obtained in 94% purity by the disclosed process. WO '435 discloses a process for preparing the 5-phenyl-1-trityl-1H-tetrazole by combining 5-phenyl-1-tributyltin-tetrazole with aqueous sodium hydroxide to produce 5-phenyl-1H-tetrazole and combining the 5-phenyl-1H-tetrazole with chlorotriphenylmethane in toluene to produce 5-phenyl-1-trityl-1H-tetrazole. See WO '435, p. 15 (scheme 1). The tributyl tin starting material is undesirable because the tin can be carried over into the 5-phenyl-1-trityl-1H-tetrazole, and to the API irbesartan prepared from the 5-phenyl-1-trityl-1H-tetrazole. The tin is difficult to remove from the API irbesartan.

There is a need in the art for an improved method for the synthesis of 5-phenyl-1-trityl-1H-tetrazole.

### SUMMARY OF THE INVENTION

In one embodiment, the invention encompasses a process for preparing 5-phenyl-1-trityl-1H-tetrazole comprising reacting 5-phenyl-1H-tetrazole with a compound of the formula C(C₆H₅)₃—R in the presence of at least one base, at least one phase transfer catalyst, water and at least one organic solvent, thereby forming a reaction mixture having at least an organic phase and an aqueous phase, wherein R is a leaving group.

In another embodiment, the invention encompasses a process for preparing irbesartan comprising preparing 5-phenyl-1-trityl-1H-tetrazole by the above-described process, and converting the 5-phenyl-1-trityl-1H-tetrazole into irbesartan. The 5-phenyl-1-trityl-1H-tetrazole may be converted into irbesartan by a process comprising: a) converting the 5-phenyl-1-trityl-1H-tetrazole into 2-[5-(1-trityl-1H-tetrazol)phenylboronic acid]; b) converting the 2-[5-(1-trityl-1H-tetrazol)phenylboronic acid into trityl irbesartan; and c) converting the trityl irbesartan into irbesartan.

### DETAILED DESCRIPTION OF THE INVENTION

The invention provides an improved process for the preparation of the irbesartan intermediate 5-phenyl-1-trityl-1H-tetrazole of formula (II).

The time periods described herein are time periods suitable for laboratory-scale preparations. One of ordinary skill in the art understands that suitable time periods will vary based upon the amounts of reagents present, and can adjust the time periods accordingly.

In one embodiment, the invention encompasses a process for preparing 5-phenyl-1-trityl-1H-tetrazole (“compound II”) comprising reacting 5-phenyl-1H-tetrazole (“compound III”) with a compound of the formula C(C₆H₅)₃—R (“compound IV”) in, the presence of at least one base, at least one phase transfer catalyst, water, and at least one organic solvent, thereby forming a reaction mixture having at least an organic phase and an aqueous phase, wherein R is a leaving group. The process may be illustrated by the following Scheme 1. 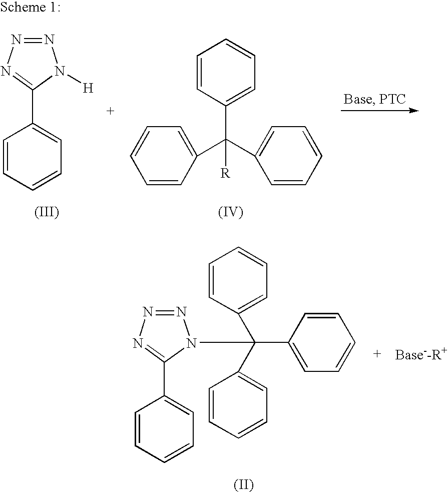
wherein Base⁻R⁺ is a by-product salt produced from the combination of the anion derived from the base and the leaving group R. Preferably, the leaving group is a halide or a tosyl group. One advantage of employing a multi-phasic reaction system is that at the end of the reaction, the desired 5-phenyl-1-trityl-1H-tetrazole product is dissolved primarily in the organic phase, while the by-product salt is dissolved primarily in the aqueous phase. Accordingly, one can isolate the desired product from the by-product salt simply by separating the aqueous and organic phases, thus, eliminating the need for additional purification steps, such as those disclosed in the above-described prior art.

Typically, the reaction mixture has at least an aqueous and an organic phase. The reaction mixture may initially be monophasic, but gradually will separate into at least two phases during the progress of the reaction so that it has at least an aqueous and an organic phase by the time the reaction is complete. The invention also encompasses multi-phasic reaction mixtures that have at least a solid phase, an aqueous phase, and an organic phase. Preferably, the reaction mixture is biphasic from the beginning of the reaction and remains biphasic through to the completion of the reaction. Preferably, water is present in the mixture in an amount of from about 2 milliliters to about 10 milliliters per gram of the 5-phenyl-1H-tetrazole.

Typically, the organic solvent is present in an amount of about 5 milliliters to about 20 milliliters per gram of 5-phenyl-1H-tetrazole. Typically, the organic solvent is a water-immiscible solvent, and preferably an aprotic organic water-immiscible solvent. Examples of suitable aprotic organic water-immiscible solvents may include, but are not limited to, nitriles, ethers, aromatic compounds, halogenated solvents, esters, and ketones. Preferably, the nitrile is a C₂₋₆ nitrile, and more preferably benzonitrile. Preferably, the ether is a C₄₋₆ ether, and more preferably dimethoxyethane. Preferably, the aromatic compound is a C₆₋₈ aromatic compound, and more preferably benzene, toluene, xylene, or ethyl benzene. Preferably, the halogenated solvent is a C₁₋₃ halogenated solvent, and more preferably dichloroethane, chloroform, or dichloromethane. Preferably, the ester is a C₂₋₄ ester, and more preferably ethyl acetate or butyl acetate. Preferably, the ketone is a C₃₋₆ ketone, and more preferably methylethylketone or cyclohexanone. Preferred aprotic organic solvents are selected from the group consisting of halogenated solvents and aromatic compounds. More preferred aprotic organic solvents are selected from the group consisting of dichloromethane, chloroform, xylene, ethyl benzene, and toluene. Most preferred aprotic organic solvents are selected from the group consisting of toluene, xylene, choroform, and dichloromethane.

The base may be an inorganic or an organic base, and is preferably an inorganic base. Preferred organic bases include, but are not limited to, tertiary amines. Preferred inorganic bases include, but are not limited to, alkali and alkaline earth metal bases. Preferably, the alkali or alkaline earth metal base is selected from the group consisting of sodium hydroxide, potassium hydroxide, lithium hydroxide, sodium carbonate, sodium bicarbonate, potassium carbonate, potassium bicarbonate, lithium carbonate, and lithium bicarbonate. More preferably, the base is sodium carbonate. The base is typically present in an amount of about 1.2 to about 2.0 moles per mole of the 5-phenyl-1H-tetrazole.
Preferably, the compound IV is a triphenylmethyl halide or tosylate. Preferably, the halide is bromine, chlorine, fluorine, or iodine, and more preferably chlorine. Preferably, the tosyl is ortho-tosyl, tosyl chloride, or tosyl bromide. Typically, the compound IV is present in an amount of about 0.8 moles to about 1.5 moles per mole of the 5-phenyl-1H-tetrazole.

The phase transfer catalyst should be present in a sufficient amount to solubilize the C(C₆H₅)₃—R in the aqueous phase, thus, preferably facilitating the reaction between the (C₆H₅)₃—R and compound III, which is soluble in the aqueous phase. Suitable phase transfer catalysts and suitable quantities of such catalysts would be known to one skilled in the art and include, but are not limited to, quaternary ammonium salts, phosphonium salts, crown ethers, and pyridinium salts.

Examples of suitable quaternary ammonium salts include, but are not limited to salts of the formula R′₄N⁺X⁻ and R′₄N⁺OH⁻, wherein R′ is an alkyl or aryl and X is a halogen. Preferably, the alkyl is a C₁₋₆ alkyl. Preferably, the aryl is a C₆₋₁₀ aryl. Preferably, the halogen is chlorine, bromine, fluorine, or iodine, and more preferably chlorine or bromine. Preferred salts of the formula R′₄N⁺X⁻ include tetraalkylammonium chlorides, such as tetramethylammonium chloride, tetraethylammonium chloride, tetrapropylammonium, chloride and tetrabutylammonium chloride (“TBAC”); tetraalkylammonioum bromides, such as tetramethylammonium bromide, tetraethylammonium bromide, tetrapropylammonium bromide and tetrabutylammonium bromide; benzyltrialkylammonium halides, such as benzyltrimethylammonium chloride, benzyltrimethylammonium bromide, benzyl-tri-n-butylammonium chloride (“BTBAC”) and benzyl-tri-n-butylammonium bromide; cetyltrialkylammonium halides, such as cetyltrimethylammonium chloride, cetyltrimethylammonium bromide, cetyltriethylammonium chloride and cetyltriethylammonium bromide. Preferred salts of the formula include R′₄N⁺OH⁻ tetraalkylammonium hydroxides, such as tetramethylammonium hydroxide, tetraethylammonium hydroxide, tetrapropylammonium hydroxide and tetrabutylammonium hydroxide; and benzyltrialkylammonium hydroxides, such as benzyltrimethylammonium hydroxide, benayltrimethylammonium hydroxide, benzyltri-n-butylammonium hydroxide and benzyl-tri-n-butylammonium hydroxide.

Examples of suitable phosphonium salts include, but are not limited to, salts of the formula R′₃P⁺X⁻, wherein R′ is an alkyl or aryl and X is a halogen. Preferably, the halogen is chlorine, bromine, fluorine, or iodine. Preferably, the alkyl is a C₁₋₆ alkyl. Preferably, the aryl is a C₆₋₁₀ aryl. Preferred salts of the formula R′₃P⁺X⁻ include phosphonium chloride, phosphonium bromide, trimethylphosphonium chloride, triethylphosphonium bromide, tetramethylphosphonium chloride, tetramethylphosphonium bromide, ethyl triphenyl phosphonium bromide, ethyl triphenyl phosphonium iodide, butyl triphenyl phosphonium bromide, benzyl triphenyl phosphonium chloride, methyl triphenyl phosphonium bromide, methyl triphenyl phosphonium iodide, tetraphenyl phosphonium bromide, methyl triphenyl phosphonium bromide, butyl triphenyl phosphonium chloride, (methoxy methyl) triphenyl phosphonium chloride, and phosphonium iodide.

Examples of suitable crown ethers include, but are not limited to, 8-crown-6, and 15-crown-5.

Examples of suitable pyridinium salts include, but are not limited to, salts of the formula 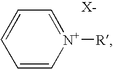
wherein R′ is an alkyl or aryl and X is a halogen. Preferably, the alkyl is a C₁₋₆ alkyl. Preferably, the aryl is a C₆₋₁₀ aryl. Preferred salts of the formula 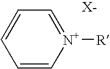
include cetyl pyridinium chloride, cetyl pyridinium bromide, lauryl pyridinium chloride, and dodecyl pyridinium chloride.

Preferably, the phase transfer catalyst is a quaternary ammonium salt. Quaternary ammonium salts are readily available commercially and allow one to produce the desired product in high yield. More preferably, the phase transfer catalyst is selected from the group consisting of tetraalkylammonium halides, benzyltrialkylammonium halides, and tetraalkylammonium hydrogen sulfate. Preferably, the phase transfer catalyst is present in an amount of about 0.0001 mole to about 1 mole per mole of the 5-phenyl-1H-tetrazole, and more preferably about 0.01 mole to about 0.05 mole of the 5-phenyl-1H-tetrazole.

Preferably, the 5-phenyl-1H-tetrazole and the compound of the formula C(C₆H₅)₃—R are reacted, preferably with agitation, for about 30 minutes to about 10 hours, and more preferably for about 3 to about 4 hours. Preferably, the 5-phenyl-1H-tetrazole and the compound of the formula C(C₆H₅)₃—R are reacted at a temperature of about 0° C. to about 40° C., more preferably at a temperature of about 0° C. to about 25° C., and most preferably at 0° C. to about 5° C.

The progress of the reaction may be monitored by HPLC and/or TLC.

In a preferred embodiment, the process comprises combining the 5-phenyl-1H-tetazole, the base, and the water to form a first mixture, combining the first mixture with the phase transfer catalyst to obtain a second mixture, and combining the second mixture with a solution of the compound of the formula C(C₆H₅)₃—R in the organic solvent to obtain a biphasic reaction mixture. Preferably, the solution of the compound of the formula C(C₆H₅)₃—R in the organic solvent is added drop-wise to the second mixture, more preferably over a period of about 10 minutes to about 60 minutes.

The resulting 5-phenyl-1′-trityl-1H-tetrazole may be recovered from the reaction mixture by any method known to one of ordinary skill in the art. Such methods include, but are not limited to, separating the organic phase from the reaction mixture, and removing the solvent from the organic phase, preferably by distillation, to obtain a residue or suspension of 5-phenyl-1-trityl-1H-tetrazole.

Optionally, the 5-phenyl-1-trityl-1H-tetrazole may be purified from the residue. Residual organic solvent may be removed from the 5-phenyl-1-trityl-1H-tetrazole by adding to the residue a solvent capable of forming an azeotrope with the organic solvent, and removing the azeotrope from the residue, preferably by distillation. When the organic solvent is chloroform, toluene, xylene, and/or dichloromethane, the solvent capable of forming an azeotrope with the organic solvent is preferably a C₁₋₃ nitrile, and more preferably acetonitrile. The 5-phenyl-1-trityl-1H-tetrazole may then be further purified by crystallization from the solvent capable of forming an azeotrope with the organic solvent.

Preferably, the crystallized 5-phenyl-1-trityl-1H-tetrazole produced by the above-described process has a purity of at least 95%, more preferably at least 97% and most preferably at least 98% area by HPLC.

The invention also encompasses a process for preparing irbesartan comprising preparing 5-phenyl-1-trityl-1H-tetrazole by the above-described process, and converting the 5-phenyl-1-trityl-1H-tetrazole into irbesartan.

The 5-phenyl-1-trityl-1H-tetrazole may be converted into irbesartan by any method known to one of ordinary skill in the art, including, for example, any of the methods disclosed in WO 2004/065383, U.S. Pat. No. 5,270,317, or U.S. Pat. No. 5,559,233, all of which are incorporated herein by reference. Typically, 5-phenyl-1-trityl-1H-tetrazole is converted to irbesartan by a process comprising reacting the 5-phenyl-1-trityl-1H-tetrazole with a borate in the presence of a base to obtain 2-[5-(1-trityl-1H-tetrazol) phenylboronic acid]; reacting the 2-[5-(1-trityl-1H-tetrazol) phenylboronic acid] with 2-butyl-3(4′-bromobenzyl)-1,3-diazaspiro[4,4]non-1-ene-4-one in the presence of at least one catalyst to obtain 2-butyl-3[2′-(triphenylmethyltetrazol-5-yl)-biphenyl-4-yl methyl]-1,3-diazaspiro[4, 4]non-1-ene-4-one (“trityl irbesartan”); and converting the trityl irbesartan to irbesartan by acid hydrolysis. See, e.g., WO '383, pp. 12-15. The process may be illustrated by the following Scheme 2.

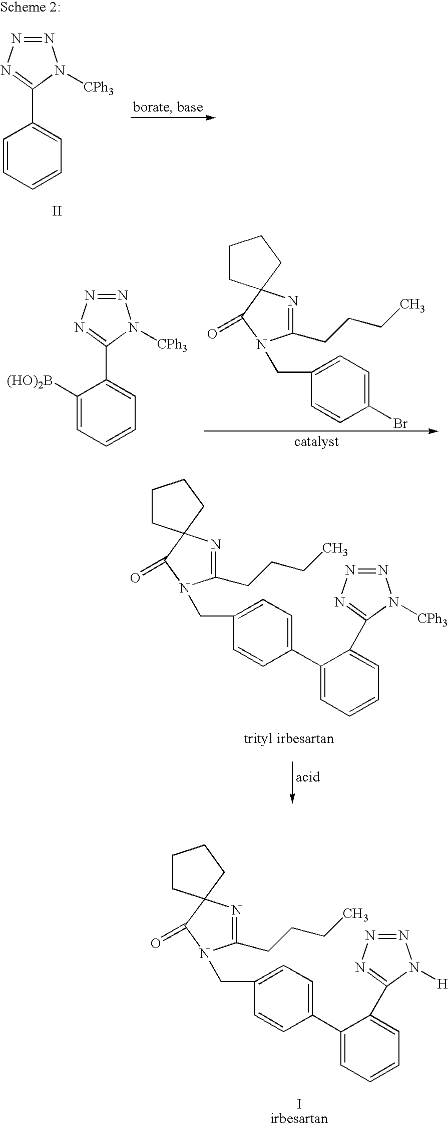

Typically, the 2-[5-(1-trityl-1H-tetrazol) phenylboronic acid] is reacted with the 2-butyl-3(4′-bromobenzyl)-1,3-diazaspiro[4,4]non-1-ene-4-one in the presence of a solvent and at least one catalyst to obtain the trityl irbesartan. Preferably, the solvent is present in an amount of about 5 milliliters to about 25 milliliters per gram of the 2-butyl-3(4′-bromobenzyl)-1,3-diazaspiro[4,4]non-1-ene-4-one. Preferably, the catalyst is present in an amount of about 1 to about 3.5 mol % relative to the amount of 2-butyl-3(4′-bromobenzyl)-1,3-diazaspiro[4,4]non-1-ene-4-one. The reaction mixture may be heated to obtain the trityl irbesartan. Preferably, the reaction mixture is heated at a temperature of about 50° C. to about 105° C., and more preferably at about reflux temperature of the solvent.

Having described the invention with reference to certain preferred embodiments, other embodiments will become apparent to one of skill in the art from consideration of the specification. The invention is further defined by reference to the following non-limiting examples describing in detail the synthesis of 5-phenyl-1-trityl-1H-tetrazole, as well as the conversion of 5-phenyl-1-trityl-1H-tetrazole to irbesartan. It will be apparent to those of skill in the art that many modifications, both to materials and methods, may be practiced without departing from the scope of the invention.

### EXAMPLES

### Preparation of 5-phenyl-1-trityl-1H-tetrazole (“compound II”)

HPLC Method for Monitoring Reaction Progress

In the following Examples 1-6, the preparation of 5-phenyl-1-trityl-1H-tetrazole from 5-phenyl-1-H-tetrazole was monitored by HPLC under the following conditions:

A YMC pack ODS-AQ column was used. The flow rate was 1.0 ml/min, the column temperature was 40° C. and the samples were detected with an ultraviolet detector at 235 nm. The sample volume was 10 μl and the samples were diluted with an 80:20 mixture of acetonitrile:methanol. The auto-sampler was set at a temperature of 5° C. Samples were gradient eluted from the column with a mixture of buffer and methanol. The buffer was prepared by dissolving 1.36 grams of KH₂PO₄ in 1000 ml of water, and adjusting the pH of the solution to 2.5±0.05 with 10% v/v orthophosphoric acid solution. The gradient was as follows:

| Time (minutes) | % Buffer | % Methanol |
|---|---|---|
| 0 | 40 | 60 |
| 5.0 | 40 | 60 |
| 15 | 13 | 87 |
| 30 | 13 | 87 |
| 31 | 40 | 60 |
| 37 | 40 | 60 |

Under these conditions, the starting 5-phenyl-1-H-tetrazole had a retention time of 4.165 minutes and the product 5-phenyl-1-trityl-1H-tetrazole had a retention time of 23.60 minutes. The starting 5-phenyl-1H-tetrazole also had a relative retention time of 0.176.

### Example 1

### Preparation of 5-phenyl-1-trityl-1H-tetrazole

A mixture of 5-phenyl-1H-tetrazole (100.0 g, 0.68 mol) and sodium carbonate (110.84 g, 1.03 mol) in de-mineralized (“D.M.”) water (1000 ml) was cooled to 0 to 5° C. in a round-bottomed flask. Tetrabutyl ammonium bromide (5.0 g, 0.015 mol) was then added to the flask at 0 to 5° C. A solution of trityl chloride (228.88 g, 0.82 mol) in chloroform (1250 ml) was then added drop-wise to the flask, while maintaining the temperature at 0 to 5° C., to form a reaction mixture. The reaction mixture was then stirred for 3-4 hrs at 0 to 5° C. and the progress of the reaction was monitored by HPLC and/or TLC. The reaction mixture was allowed to warm to room temperature after completion of the reaction.

The organic layer was separated and the upper aqueous layer was extracted with fresh chloroform (200.0 ml). The organic layers were combined and washed with fresh D.M. water (200.0 ml). The chloroform was then distilled from the combined organic layer at 45° C., until a solid began to form. Acetonitrile (1000 ml) was then added and distillation of 10% of the acetonitrile was performed to remove remaining traces of chloroform. The remaining organic layer was then stirred at room temperature for 5 to 6 hrs and then cooled to 0 to 5° C. The temperature was maintained at 0 to 5° C. for 60-90 minutes with stirring. The reaction mass was then filtered from the organic layer, washed with fresh chilled acetonitrile (100.0 ml), and dried under reduced pressure of about 500-760 mm Hg at 40-45° C. [Yield: 97.66%; Purity: 99.13% area by HPLC]

### Example 2

### Preparation of 5-phenyl-1-trityl-1H-tetrazole

A mixture of 5-phenyl-1H-tetrazole (100.0 g, 0.68 mol, obtained from Taizhounova Medicine Chem. Co. (China)) and sodium carbonate (110.84 g, 1.03 mol) in D.M. water (1000 ml) was cooled to 0 to 5° C. in a round-bottomed flask. Tetrabutyl ammonium bromide (5.0 g, 0.015 mol) was then added to the flask at 0 to 5° C. A solution of trityl chloride (228.88 g, 0.82 mol) in toluene (1250 ml) was then added drop-wise to the flask while maintaining the temperature at 0 to 5° C., to form a reaction mixture. The reaction mixture was stirred for 3-4 hrs at 0 to 5° C. and the reaction progress was monitored by HPLC and TLC. The product precipitated during the reaction. The reaction mixture was allowed to warm to room temperature after the completion of the reaction. The product dissolved in the organic layer of the reaction mixture upon warming to room temperature.

The organic layer was separated and the upper aqueous layer was extracted with fresh toluene (200.0 ml). The organic layers were combined and washed with fresh D.M. water (200.0 ml). The toluene was then distilled from the combined organic layer at 65° C. Acetonitrile (1000 ml) was then added and distillation of 10% of the acetonitrile was performed to remove remaining traces of toluene. The remaining organic layer was then stirred at room temperature for 5 to 6 hrs and then cooled to 0 to 5° C. The temperature was maintained at 0 to 5° C. for 60-90 minutes with stirring. The reaction mass was then filtered from the organic layer, washed with fresh chilled acetonitrile (100.0 ml), and dried under vacuum at a pressure of about 500-760 mm Hg at 45° C. [Yield: 67.0%; Purity: 98% area by HPLC]

### Example 3

### Preparation of 5-phenyl-1-trityl-1H-tetrazole

A mixture of 5-phenyl-1H-tetrazole (100.0 g, 0.68 mol) and sodium carbonate (110.84 g, 1.03 mol) in D.M. water (1000 ml) was cooled to 0 to 5° C. in a four-necked, three-liter, round-bottomed flask. Tetrabutyl ammonium bromide (5.0 g, 0.015 mol) was then added to the flask at 0 to 5° C. A solution of trityl chloride (228.88 g, 0.82 mol) in xylene (1250 ml) was then added drop-wise to the flask while maintaining the temperature at 0 to 5° C., to form a reaction mixture. The reaction mixture was stirred for 3-4 hrs at 0 to 5° C. and the reaction progress was monitored by HPLC and TLC. The product precipitated during the reaction. The reaction mixture was then allowed to warm to room temperature after the completion of the reaction. The product dissolved in the organic layer of the reaction mixture upon warming to room temperature.

The organic layer was separated and the upper aqueous layer was extracted with fresh xylene (200.0 ml). The organic layers were combined and washed with fresh D.M. water (200.0 ml). The xylene was then distilled from the combined organic layer at 65° C. Acetonitrile (1000 ml) was then added and distillation of 10% of the acetonitrile was performed to remove remaining traces of xylene. The remaining organic layer was then stirred at room temperature for 5 to 6 hrs and then cooled to 0 to 5° C. The temperature was maintained at 0 to 5° C. for 60-90 minutes with stirring. The reaction mass was then filtered from the organic layer, washed with fresh chilled acetonitrile (100.0 ml), and dried under vacuum at a pressure of about 500-760 mm Hg at 45° C. [Yield: 67.5%; Purity: 97.4% area by HPLC]

### Example 4

### Preparation of 5-phenyl-1-trityl-1H-tetrazole

A mixture of 5-phenyl-1H-tetrazole (100.0 g, 0.68 mol) and sodium carbonate (110.84 g, 1.03 mol) in D.M. water (1000 ml) was cooled to 0 to 5° C. in a round-bottomed flask. Tetrabutyl ammonium hydrogen sulfate (5.3 g, 0.015 mol) was then added to the flask at 0 to 5° C. A solution of trityl chloride (228.88 g, 0.82 mol) in dichloromethane (1250 ml) was then added drop-wise to the flask while maintaining the temperature at 0 to 5° C., to form a reaction mixture. The reaction mixture was then stirred for 3-4 hrs at 0 to 5° C. and the reaction progress was monitored by HPLC and TLC. The reaction mixture was then allowed to warm to room temperature after the completion of the reaction.

The organic layer was separated and the upper aqueous layer was extracted with fresh dichloromethane (200.0 ml). The organic layers were combined and washed with fresh D.M. water (200.0 ml). The dichloromethane was then distilled from the combined organic layer at 45° C. Acetonitrile (1000 ml) was then added and distillation of 10% of the acetonitrile was performed to remove remaining traces of dichloromethane. The remaining organic layer was then stirred at room temperature for 5 to 6 hrs and then cooled to 0 to 5° C. The temperature was maintained at 0 to 5° C. for 60-90 minutes with stirring. The reaction mass was then filtered from the organic layer, washed with fresh chilled acetonitrile (100.0 ml), and dried under vacuum at a pressure of about 500-760 mm Hg at 45° C. [Yield: 97.5%; Purity: 99.6% area by HPLC]

### Example 5

### Preparation of 5-phenyl-1-trityl-1H-tetrazole

A mixture of 5-phenyl-1H-tetrazole (100.0 g, 0.68 mol) and sodium carbonate (110.84 g, 1.03 mol) in D.M. water (1000 ml) was cooled to 0 to 5° C. in a four-necked, three-liter, round-bottomed flask. Tetraheptyl ammonium bromide (7.6 g, 0.015 mol) was then added to the flask at 0 to 5° C. A solution of trityl chloride (228.88 g, 0.82 mol) in dichloromethane (1250 ml) was then added drop-wise to the flask while maintaining the temperature at 0 to 5° C., to form a reaction mixture. The reaction mixture was then stirred for 3-4 hrs at 0 to 5° C. and the reaction progress was monitored by HPLC and TLC. The reaction mixture was then allowed to warm to room temperature after completion of the reaction.

The organic layer was then separated and the upper aqueous layer was extracted with fresh dichloromethane (200.0 ml). The organic layers were combined and washed with fresh D.M. water (200.0 ml). The dichloromethane was then distilled from the combined organic layer at 45° C., until a solid began to form. Acetonitrile (1000 ml) was then added and distillation of 10% of the acetonitrile was performed to remove remaining traces of dichloromethane. The remaining organic layer was then stirred at room temperature for 5 to 6 hrs and then cooled to 0 to 5° C. The temperature was maintained at 0 to 5° C. for 60-90 minutes with stirring. The reaction mass was then filtered from the organic layer, washed with fresh chilled acetonitrile (100.0 ml), and dried under vacuum at a pressure of about 500-760 mm Hg at 45° C. [Yield: 95.9%; Purity: 98.1% area by HPLC]

### Example 6

### Preparation of 5-phenyl-1-trityl-1H-tetrazole

A mixture of 5-phenyl-1H-tetrazole (100.0 g, 0.68 mol) and sodium carbonate (110.84 g, 1.03 mol) in D.M. water (1000 ml) was cooled to 0 to 5° C. in a four-necked, three-liter, round-bottomed flask. Benzyltributyl ammonium chloride (0.49 g, 0.015 mol) was then added to the flask at 0 to 5° C. A solution of trityl chloride (228.88 g, 0.82 mol) in dichloromethane (1250 ml) was then added drop-wise to the flask while maintaining the temperature at 0 to 5° C., to form a reaction mixture. The reaction mixture was stirred for 3-4 hrs at 0 to 5° C. and the reaction progress was monitored by HPLC and TLC. The reaction mixture was allowed to warm to room temperature after the completion of the reaction.

The organic layer was then separated and the upper aqueous layer was extracted with fresh dichloromethane (200.0 ml). The organic layers were combined and washed with fresh D.M. water (200.0 ml). The dichloromethane was then distilled from the combined organic layer at 45° C., until a solid began to form. Acetonitrile (1000 ml) was then added and distillation of 10% of the acetonitrile was performed to remove remaining traces of dichloromethane. The remaining organic layer was then stirred at room temperature for 5 to 6 hrs and then cooled to 0 to 5° C. The temperature was maintained at 0 to 5° C. for 60-90 minutes with stirring. The reaction mass was then filtered from the organic layer, washed with fresh chilled acetonitrile (100.0 ml), and dried under vacuum at a pressure of about 500-760 mm Hg at 45° C. [Yield: 93.1%; Purity: 97.6% area by HPLC]

### Preparation of 2-[5-(1-trityl-1H-tetrazol)phenylboronic acid] from 5-phenyl-1-trityl-1H-tetrazole

HPLC Method for Monitoring Reaction Progress:

In the following Examples 7-10, the preparation of 2-[5-(1-trityl-1H-tetrazol)phenylboronic acid] from 5-phenyl-1-trityl-1H-tetrazole was monitored by HPLC under the following conditions:

A YMC pack ODS-AQ column was used. The flow rate was 1.0 ml/min, the column temperature was 40° C. and the samples were detected with an ultraviolet detector at 235 nm. The sample volume was 10 μl and the samples were diluted with an 80:20 mixture of acetonitrile:methanol. The auto-sampler was set at a temperature of 5° C. Samples were gradient eluted from the column with a mixture of buffer and methanol. The buffer was prepared by dissolving 1.36 grams of KH₂PO₄ in 1000 ml of water, and adjusting the pH of the solution to 2.5±0.05 with 10% v/v orthophosphoric acid solution. The gradient was as follows:

| Time (minutes) | % Buffer | % Methanol |
|---|---|---|
| 0 | 40 | 60 |
| 5.0 | 40 | 60 |
| 15 | 13 | 87 |
| 30 | 13 | 87 |
| 31 | 40 | 60 |
| 37 | 40 | 60 |

Under these conditions, the starting 5-phenyl-1-trityl-1H-tetrazole had a retention time of 23.654 minutes and the product 2-[5-(1-trityl-1H-tetrazol)phenylboronic acid] had a retention time of 19.55 minutes. The starting 5-phenyl-1-trityl-1H-tetrazole also had a relative retention time of 1.20.

### Example 7

### Preparation of 2-[5-(1-trityl-1H-tetrazol) phenylboronic acid]

A solution of 5-phenyl-1-trityl-1H-tetrazole (100.0 g, 0.26 mol) in dry tetrahydrofuran (“THF”) (800.0 ml) was cooled to about −25° C. under nitrogen or argon. n-Butyl lithium (1.6M in hexane) was added to quench the traces of water in the reaction mixture until the color of the reaction mixture remained red for at least 5 minutes. n-Butyl lithium (180.0 ml) was then added dropwise to the reaction mixture over a period of about 45-50 minutes at a temperature below −25° C. The temperature of the reaction mixture was raised to −5° C. The reaction mixture was stirred at −5° C. for 3 hrs. The reaction mixture was cooled to −25° C. and triisopropyl borate (101.0 ml) was added. The reaction mixture was warmed to a temperature of about 25-35° C. and the reaction mixture was stirred for 2 hrs. The reaction mixture was cooled again to 0-5° C. and 3% aq. acetic acid (667.0 ml) was slowly added over 30-40 minutes. The reaction mixture was stirred for 30-40 minutes and then filtered. The product was washed with 500 ml water. The wet material was dried at about 45° C. under reduced pressure of about 500-760 mm Hg until constant weight. [Yield: 94%; Purity: 94% area by HPLC]

### Example 8

### Preparation of 2-[5-(1-trityl-1H-tetrazol) phenylboronic acid]

A solution of 5-phenyl-1H-tetrazole (100.0 g, 0.26 mol) in dry THF (800.0 ml) was cooled to −25° C. under nitrogen or argon. n-Hexyl lithium (2.1M in hexane) was added to quenched the traces of water in reaction until the color of reaction remained red at least for 5 minutes. n-Hexyl lithium (150.0 ml) was then added drop-wise over a period of 45-50 minutes at a temperature below −25° C. The temperature of the reaction mixture was raised to −5° C. The reaction mixture was stirred at −5° C. for 3 hrs. The reaction mixture was cooled to −25° C. and triisopropyl borate (101.0 ml) was added. The reaction mixture was warmed to a temperature of about 25-35° C. and the reaction mixture was stirred for 2 hrs. The reaction mixture was cooled again to 0-5° C. and 3% aq. acetic acid (667.0 ml) was added drop-wise over 30-40 minutes. The reaction mixture was stirred for 30-40 minutes and then filtered to isolate the product. The product was washed with 500 ml water. The wet material was dried at about 45° C. under reduced pressure of about 500-760 mm Hg until constant weight. [Yield: 94%; Purity: 94% area by HPLC]

### Example 9

### Preparation of 2-[5-(1-trityl-1H-tetrazol) phenylboronic acid]

A solution of 5-phenyl-1H-tetrazole (100.0 g, 0.26 mol) in dry 2-methyl tetrahydrofuran (1000.0 ml) was cooled to −25° C. under nitrogen or argon. n-Butyl lithium (1.6M in hexane) was added to quench the traces of water in the reaction mixture until the color of the reaction mixture remained red for at least 5 minutes. n-Butyl lithium (180.0 ml) was then added drop-wise to the reaction mixture over a period of 45-50 minutes at a temperature below −25° C. The temperature of the reaction mixture was raised to −5° C. The reaction mixture was stirred at −5° C. for 3 hrs. The reaction mixture was cooled to −25° C. and triisopropyl borate (101.0 ml) was added. The reaction mixture was warmed to a temperature of about 25-35° C. and the reaction mixture was stirred for 2 hrs. The reaction mixture was cooled again to 0-5° C. and 3% aq. acetic acid (667.0 ml) was added drop-wise over 30-40 minutes. The reaction mixture was stirred for 30-40 minutes and then filtered. The product was washed with 500 ml water. The wet material was dried at about 45° C. under reduced pressure of about 500-760 mm Hg until constant weight. [Yield: 94%; Purity: 90% area by HPLC]

### Example 10

### Preparation of 2-[5-(1-trityl-1H-tetrazol) phenylboronic acid]

A solution of 5-phenyl-1H-tetrazole (100.0 g, 0.26 mol) in dry 2-methyl tetrahydrofuran (1000.0 ml) was cooled to −25° C. under nitrogen or argon. n-Hexyl lithium (2.1M in hexane) was added to quench the traces of water in the reaction mixture until the color of the reaction mixture remained red for at least 5 minutes. The n-Hexyl lithium (150.0 ml) was then added drop-wise to the reaction mixture over a period of 45-50 minutes at a temperature below −25° C. The temperature of the reaction mixture was raised to −5° C. The reaction mixture was stirred at −5° C. for 3 hrs. The reaction mixture was cooled to −25° C. and triisopropyl borate (101.0 ml) was added. The reaction mixture was warmed to a temperature of about 25-35° C. and the reaction mixture was stirred for 2 hrs. The reaction mixture was cooled again to 0-5° C. and 3% aq. acetic acid (667.0 ml) was added drop-wise over 30-40 minutes. The reaction mixture was stirred for 30-40 minutes and then filtered. The product was washed with 500 ml water. The wet material was dried at about 45° C. under reduced pressure of about 500-760 mm Hg until constant weight. [Yield: 94%; Purity: 92% area by HPLC]

### Preparation of trityl irbesartan from 2-[5-(1-trityl-1H-tetrazol)phenylboronic acid]

HPLC Method for Monitoring Reaction Progress:

In the following Examples 11-16, the preparation of trityl irbesartan from 2-[5-(1-trityl-1H-tetrazol)phenylboronic acid] and 2-butyl-3(4′-bromobenzyl)-1,3-diazaspiro[4,4]non-1-ene-4-one was monitored by HPLC under the following conditions:

A YMC pack ODS-AQ column was used. The flow rate was 1.0 ml/min, the column temperature was 40° C. and the samples were detected with an ultraviolet detector at 235 nm. The sample volume was 10 μl and the samples were diluted with an 80:20 mixture of acetonitrile:methanol. The auto-sampler was set at a temperature of 5° C. Samples were gradient eluted from the column with a mixture of buffer and methanol. The buffer was prepared by dissolving 1.36 grams of KH₂PO₄ in 1000 ml of water, and adjusting the pH of the solution to 2.5±0.05 with 10% v/v orthophosphoric acid solution. The gradient was as follows:

| Time (minutes) | % Buffer | % Methanol |
|---|---|---|
| 0 | 40 | 60 |
| 5.0 | 40 | 60 |
| 15 | 13 | 87 |
| 30 | 13 | 87 |
| 31 | 40 | 60 |
| 37 | 40 | 60 |

Under these conditions, the starting 2-[5-(1-trityl-1H-tetrazol)phenylboronic acid] had a retention time of 19.55 minutes, the starting 2-butyl-3(4′-bromobenzyl)-1,3-diazaspiro[4,4]non-1-ene-4-one had a retention time of 17.42 minutes and the product trityl irbesartan had a retention time of 30.86 minutes. The starting 2-[5-(1-trityl-1H-tetrazol)phenylboronic acid] and 2-butyl-3(4′-bromobenzyl)-1,3-diazaspiro[4,4]non-1-ene-4-one also had relative retention times of 0.63 and 0.56, respectively.

### Example 11

### Preparation of 2-butyl-3[2′-(triphenylmethyltetrazol-5-yl)-biphenyl-4-ylmethyl]-1,3-diazaspiro[4, 4]non-1-ene-4-one (“trityl irbesartan”)

A mixture of triphenyl phosphine (5.42 g, 0.02 mol) in toluene (1000.0 ml) was degassed by purging with argon gas for 25 minutes while stirring at 25-35° C., and then Pd(OAc)₂ (0.9268 g, 0.004 mol) was added in one lot. The reaction mixture was stirred for 30 minutes. 2-[5-(1-trityl-1H-tetrazol) phenylboronic acid] (149.76 g, 0.29 mol) was added and the reaction mixture was stirred for 10 minutes. D.M water (12.38 ml, 0.69 mol) was added and the reaction mixture was stirred for 30 minutes. Potassium carbonate (224.0 g, 0.69 mol) and 2-butyl-3(4′-bromobenzyl)-1,3-diazaspiro[4,4]non-1-ene-4-one (100.0 g, 0.275 mol) was added to the reaction mixture at room temperature. The reaction mixture was refluxed at 95-100° C. for 3-4 hrs. The reaction progress was monitored by HPLC.

D.M. water (1000.0 ml) was added after completion of the reaction and the organic layer was separated. The aqueous layer was extracted with fresh toluene (250.0 ml×2). The combined organic layer was washed with fresh D.M. water (500.0 ml) and the organic layer was dried with sodium sulfate. The toluene was distilled out under vacuum at 60° C. The product was crystallized from the residue by adding isopropyl alcohol (800.0 ml) and stirring overnight at room temperature. The product was filtered and washed with fresh isopropyl alcohol (100.0 ml). The product was then dried at 60° C. under reduced pressure of about 500-760 mm Hg. [Yield: 80%; Purity: 97% area by HPLC]

### Example 12

### Preparation of 2-butyl-3[2′-(triphenylmethyltetrazol-5-yl)-biphenyl-4-yl methyl]-1,3-diazaspiro[4,4]non-1-ene-4-one (“trityl irbesartan”)

A mixture of triphenyl phosphine (5.42 g, 0.02 mol) in toluene (1000.0 ml) was degassed by purging with argon gas for 25 minutes while stirring at room temperature (25-35° C.), and then Pd(OAc)₂ (0.9268 g, 0.004 mol) was added in one lot. The reaction mixture was stirred for 30-35 minutes. 2-[5-(1-trityl-1H-tetrazol) phenylboronic acid] (149.76 g, 0.29 mol) was added and the reaction mixture was stirred for 10-15 minutes. D.M water (12.38 ml, 0.69 mol) was added and the reaction mixture was stirred for 30-35 minutes. Cesium carbonate (224.0 g, 0.69 mol) and 2-butyl-3(4′-bromobenzyl)-1,3-diazaspiro[4,4]non-1-ene-4-one (100.0 g, 0.275 mol) was added to the reaction mixture at room temperature. The reaction mixture was refluxed at 95-100° C. for 3-4 hrs. The reaction progress was monitored by HPLC.

D.M. water (1000.0 ml) was added after completion of the reaction and the organic layer was separated. The aqueous layer was extracted with fresh toluene (250.0 ml×2) with stirring. The combined organic layer was washed with fresh D.M. water (500.0 ml) and the organic layer was dried with sodium sulfate. The toluene was distilled out under vacuum at 60° C. to leave a residue. Isopropyl alcohol (200 ml) was twice added to the residue and subsequently distilled off each time. The product was then crystallized from the resulting residue by adding isopropyl alcohol (800.0 ml) and stirring overnight at room temperature. The product was filtered and washed with fresh isopropyl alcohol (100.0 ml). The product was then dried at 60° C. under vacuum at a pressure of about 500-760 mm Hg. [Yield: 80%; Purity: 97% area by HPLC]

### Example 13

### Preparation of 2-butyl-3[2′-(triphenylmethyltetrazol-5-yl)-biphenyl-4-yl methyl]-1,3-diazaspiro[4, 4]non-1-ene-4-one (“trityl irbesartan”)

A mixture of triphenyl phosphine (5.42 g, 0.02 mol) in toluene (1000.0 ml) was degassed by purging with argon gas for 25 minutes while stirring at room temperature (25-35° C.), and then Pd(OAc)₂ (0.9268 g, 0.004 mol) was added in one lot. The reaction mixture was stirred for 30-35 minutes. 2-[5-(1-trityl-1H-tetrazol) phenylboronic acid] (149.76 g, 0.29 mol) was added and the reaction mixture was stirred for 10-15 minutes. D.M water (12.38 ml, 0.69 mol) was added and the reaction mixture was stirred for 30-35 minutes. Potassium carbonate (87.0 g, 0.63 mol), potassium iodide ((9.1 g, 0.05 mol), and 2-butyl-3(4′-bromobenzyl)-1,3-diazaspiro[4,4]non-1-ene-4-one (100.0 g, 0.275 mol) was added to the reaction mixture at room temperature. The reaction mixture was refluxed at 95-100° C. for 3-4 hrs. The reaction progress was monitored by HPLC.

D.M. water (1000.0 ml) was added after completion of the reaction and the organic layer was separated. The aqueous layer was extracted with fresh toluene (250.0 ml×2) with stirring. The combined organic layer was washed with fresh D.M. water (500.0 ml) and the organic layer was dried with sodium sulfate. The toluene was distilled out under vacuum at 60° C. to leave a residue. Isopropyl alcohol (200 ml) was twice added to the residue and subsequently distilled off each time. The product was then crystallized from the resulting residue by adding isopropyl alcohol (800.0 ml) and stirring overnight at room temperature. The product was filtered and washed with fresh isopropyl alcohol (100.0 ml). The product was then dried at 60° C. under vacuum at a pressure of about 500-760 mm Hg. [Yield: 70-75%; Purity: 97% area by HPLC]

### Example 14

### Preparation of 2-butyl-3[2′-(triphenylmethyltetrazol-5-yl)-biphenyl-4-yl methyl]-1,3-diazaspiro[4, 4]non-1-ene-4-one (“trityl irbesartan”)

A mixture of triphenyl phosphine (5.42 g, 0.02 mol) in toluene (1000.0 ml) was degassed by purging with argon gas for 25 minutes while stirring at room temperature (25-35° C.), and then Pd(OAc)₂ (0.9268 g, 0.004 mol) was added in one lot. The reaction mixture was stirred for 30-35 minutes. 2-[5-(1-trityl-1H-tetrazol) phenylboronic acid] (149.76 g, 0.29 mol) was added and the reaction mixture was stirred for 10-15 minutes. D.M water (12.38 ml, 0.69 mol) was added and the reaction mixture was stirred for 30-35 minutes. Potassium hydroxide (38.5 g, 0.68 mol) and 2-butyl-3(4′-bromobenzyl)-1,3-diazaspiro[4,4]non-1-ene-4-one (100.0 g, 0.275 mol) was added to the reaction mixture at room temperature. The reaction mixture was refluxed at 95-100° C. for 3-4 hrs. The reaction progress was monitored by HPLC.

D.M. water (1000.0 ml) was added after completion of the reaction and the organic layer was separated. The aqueous layer was extracted with fresh toluene (250.0 ml×2) with stirring. The combined organic layer was washed with fresh D.M. water (500.0 ml) and the organic layer was dried with sodium sulfate. The toluene was distilled out under vacuum at 60° C. to leave a residue. Isopropyl alcohol (200 ml) was twice added to the residue and subsequently distilled off each time. The product was then crystallized from the resulting residue by adding isopropyl alcohol (800.0 ml) and stirring overnight at room temperature. The product was filtered and washed with fresh isopropyl alcohol (100.0 ml). The product was then dried at 60° C. under vacuum at a pressure of about 500-760 mm Hg. [Yield: 72-76%; Purity: 97% area by HPLC]

### Example 15

### Preparation of 2-butyl-3[2′-(triphenylmethyltetrazol-5-yl)-biphenyl-4-yl methyl]-1,3-diazaspiro[4, 4]non-1-ene-4-one (“trityl irbesartan”)

A mixture of triphenyl phosphine (5.42 g, 0.02 mol) in toluene (1000.0 ml) was degassed by purging with argon gas for 25 minutes while stirring at room temperature (25-35° C.), and then Pd(OAc)₂ (0.9268 g, 0.004 mol) was added in one lot. The reaction mixture was stirred for 30-35 minutes. 2-[5-(1-trityl-1H-tetrazol) phenylboronic acid] (149.76 g, 0.29 mol) was added and the reaction mixture was stirred for 10-15 minutes. D.M water (12.38 ml, 0.69 mol) was added and the reaction mixture was stirred for 30-35 minutes. Potassium carbonate (76.0 g, 0.55 mol), calcium hydroxide ((10.2 g, 0.13 mol), and 2-butyl-3(4′-bromobenzyl)-1,3-diazaspiro[4,4]non-1-ene-4-one (100.0 g, 0.275 mol) was added to the reaction mixture at room temperature. The reaction mixture was refluxed at 95-100° C. for 3-4 hrs. The reaction progress was monitored by HPLC.

D.M. water (1000.0 ml) was, added after completion of the reaction and the organic layer was separated. The aqueous layer was extracted with fresh toluene (250.0 ml×2) with stirring. The combined organic layer was washed with fresh D.M. water (500.0 ml) and the organic layer was dried with sodium sulfate. The toluene was distilled out under vacuum at 60° C. to leave a residue. Isopropyl alcohol (200 ml) was twice added to the residue and subsequently distilled off each time. The product was then crystallized from the resulting residue by adding isopropyl alcohol (800.0 ml) and stirring overnight at room temperature. The product was filtered and washed with fresh isopropyl alcohol (100.0 ml). The product was then dried at 60° C. under vacuum at a pressure of about 500-760 mm Hg. [Yield: 70-75%; Purity: 97% area by HPLC]

### Example 16

### Preparation of 2-butyl-3[2′-(triphenylmethyltetrazol-5-yl)-biphenyl-4-yl methyl]-1,3-diazaspiro[4, 4]non-1-ene-4-one (“trityl irbesartan”)

A mixture of triphenyl phosphine (5.42 g, 0.02 mol) in toluene (950.0 ml) and dimethylsulfoxide (“DMSO”) (50.0 ml) was degassed by purging with argon gas for 25 minutes while stirring at room temperature (25-35° C.), and then Pd(OAc)₂ (0.9268 g, 0.004 mol) was added in one lot. The reaction mixture was stirred for 30-35 minutes. 2-[5-(1-trityl-1H-tetrazol) phenylboronic acid] (149.76 g, 0.29 mol) was added and the reaction mixture was stirred for 10-15 minutes. D.M water (12.38 ml, 0.69 mol) was added and the reaction mixture was stirred for 30-35 minutes. Potassium carbonate (94.0 g, 0.68 mol) and 2-butyl-3(4′-bromobenzyl)-1,3-diazaspiro[4,4]non-1-ene-4-one (100.0 g, 0.275 mol) was added to the reaction mixture at room temperature. The reaction mixture was refluxed at 95-100° C. for 3-4 hrs. The reaction progress was monitored by HPLC.

D.M. water (1000.0 ml) was added after completion of the reaction and the organic layer was separated. The aqueous layer was extracted with fresh toluene (250.0 ml×2) with stirring. The combined organic layer was washed with fresh D.M. water (500.0 ml) and the organic layer was dried with sodium sulfate. The toluene was distilled out under vacuum at 60° C. to leave a residue. Isopropyl alcohol (200 ml) was twice added to the residue and subsequently distilled off each time. The product was then crystallized from the resulting residue by adding isopropyl alcohol (800.0 ml) and stirring overnight at room temperature. The product was filtered and washed with fresh isopropyl alcohol (100.0 ml). The product was then dried at 60° C. under vacuum at a pressure of about 500-760 mm Hg. [Yield: 70-75%; Purity: 97% area by HPLC]

### Preparation of irbesartan from trityl irbesartan

### Example 17

### Preparation of 2-butyl-3[2′-(1H-tetrazol-5-yl)-biphenyl-4-yl methyl]-1,3-diazaspiro[4, 4]non-1-ene-4-one (irbesartan)

A mixture of Trityl Irbesartan (60.0 g) in acetone (360.0 ml) and concentrated hydrochloric acid (30.8 g) was stirred in D.M. water (96.0 ml) for 1.5 to 2 hrs at 35-45° C. The progress of the reaction was monitored by TLC.

The reaction mixture was cooled to 20-25° C. by adding D.M. water (120.0 ml). The acetone was completely distilled off under vacuum and again D.M. water was added (240.0 ml). The cooled reaction mixture was basified with drop wise addition of alkali (30.0 g) solution by maintaining pH 11-13 at a temperature below 15° C. The reaction mixture was extracted with ethyl acetate (390.0 ml) at less than 15° C. The layer was adjusted to pH 3-4 at below 5° C. The isolated compound was filtered and washed with D.M. water (120.0×2 ml) and ethanol (60.0 ml). The material was unloaded and purified in ethanol. The product was dried under reduced pressure of about 500-760 mm Hg at 70-80° C. [Yield: 33.0 g; Purity: 98.75% area by HPLC]

What is claimed is:

1. A process for preparing 5-phenyl-1-trityl-1H-tetrazole comprising reacting 5-phenyl-1H-tetrazole with a compound of the formula C(C₆H₅)₃—R in the presence of at least one base, at least one phase transfer catalyst, water and at least one organic solvent, thereby forming a reaction mixture having at least an organic phase and an aqueous phase, wherein R is a leaving group.

2. The process of claim 1, wherein the leaving group is a halide or tosyl group.

3. The process of claim 2, wherein the halide is bromine, chlorine, fluorine, or iodine.

4. The process of claim 3, wherein the halide is chlorine.

5. The process of claim 2, wherein the tosyl group is ortho-tosyl, tosyl chloride, or tosyl bromide.

6. The process of claim 1, wherein the water is present in an amount of about 2 volumes to about 10 volumes per gram of the 5-phenyl-1H-tetrazole.

7. The process of claim 1, wherein the organic solvent is present in an amount of about 5 volumes to about 20 volumes per gram of 5-phenyl-1H-tetrazole.

8. The process of claim 1, wherein the organic solvent is selected from the group consisting of nitrites, ethers, aromatic compounds, halogenated solvents, esters, and ketones.

9. The process of claim 1, wherein the organic solvent is selected from the group consisting of dichloromethane, chloroform, xylene, ethyl benzene, and toluene.

10. The process of claim 1, wherein the base is an alkali or alkaline earth metal base.

11. The process of claim 10, wherein the alkali or alkaline earth metal base is selected from the group consisting of sodium hydroxide, potassium hydroxide, lithium hydroxide, sodium carbonate, sodium bicarbonate, potassium carbonate, potassium bicarbonate, lithium carbonate, and lithium bicarbonate.

12. The process of claim 1, wherein the base is present in an amount of about 1.2 to about 2.0 moles per mole of the 5-phenyl-1H-tetrazole.

13. The process of claim 1, wherein the compound of the formula C(C₆H₅)₃—R is present in an amount of about 0.8 moles to about 1.5 moles per mole of the 5-phenyl-1H-tetrazole.

14. The process of claim 1, wherein the phase transfer catalyst is selected from the group consisting of quaternary ammonium salts, phosphonium salts, crown ethers, and pyridinium salts.

15. The process of claim 14, wherein the phase transfer catalyst is a quaternary ammonium salt.

16. The process of claim 15, wherein the quaternary ammonium salt is selected from the group consisting of tetraalkylammonium halides, benzyltrialkylammonium halides, and tetraalkylammonium hydrogen sulfate.

17. The process of claim 1, wherein the phase transfer catalyst is present in an amount of about 0.0001 mole to about 1 mole per mole of the 5-phenyl-1H-tetrazole.

18. The process of claim 1, wherein the 5-phenyl-1H-tetrazole and the compound of the formula C(C₆H₅)₃—R are reacted at a temperature of about 0° C. to about 40° C.

19. The process of claim 1, wherein the 5-phenyl-1H-tetazole, the base, and the water are combined to form a first mixture; the first mixture is combined with the phase transfer catalyst to obtain a second mixture; and the second mixture with a solution of the compound of the formula C(C₆H₅)₃—R in the organic solvent to obtain a multi-phasic reaction mixture.

20. The process of claim 19, wherein the solution of the compound of the formula C(C₆H₅)₃—R in the organic solvent is added drop-wise to the second mixture.

21. The process of claim 1, further comprising recovering the 5-phenyl-1-trityl-1H-tetrazole from the organic phase.

22. The process of claim 21, further comprising: adding to the recovered 5-phenyl-1-trityl-1H-tetrazole a solvent capable of forming an azeotrope with the residual organic solvent present in the recovered 5-phenyl-1-trityl-1H-tetrazole to form a mixture; removing the azeotrope from the mixture; and, optionally, crystallizing 5-phenyl-1-trityl-1H-tetrazole from the solvent capable of forming an azeotrope with the organic solvent.

23. A process for preparing irbesartan comprising: preparing 5-phenyl-1-trityl-1H-tetrazole by the process of claim 1; and converting the 5-phenyl-1-trityl-1H-tetrazole into irbesartan.

24. The process of claim 23, wherein the 5-phenyl-1-trityl-1H-tetrazole is converted into irbesartan by a process comprising: a) converting the 5-phenyl-1-trityl-1H-tetrazole into 2-[5-(1-trityl-1H-tetrazol)phenylboronic acid]; b) converting the 2-[5-(1-trityl-1H-tetrazol)phenylboronic acid into trityl irbesartan; and c) converting the trityl irbesartan into irbesartan.
